(19) United States
(12) Patent Application Publication
(10) Pub. No.: US 2025/0009806 A1
(43) Pub. Date: Jan. 9, 2025

(54) PHARMACEUTICAL COMPOSITION COMPRISING NEURAL PROGENITOR CELLS FOR PREVENTION OR TREATMENT OF OPTIC NEUROPATHY

(71) Applicants: CHA UNIVERSITY INDUSTRY-ACADEMIC COOPERATION FOUNDATION, Pocheon-si, Gyeonggi-do (KR); SUNGKWANG MEDICAL FOUNDATION, Seoul (KR)

(72) Inventors: Helen LEW, Seoul (KR); Mi-Ra PARK, Seoul (KR); Dong-Youn HWANG, Yongin-si, Gyeonggi-do (KR); Hyun-Moon KIM, Seongnam-si, Gyeonggi-do (KR)

(73) Assignees: CHA UNIVERSITY INDUSTRY-ACADEMIC COOPERATION FOUNDATION, Pocheon-si, Gyeonggi-do (KR); SUNGKWANG MEDICAL FOUNDATION, Seoul (KR)

(21) Appl. No.: 18/715,546

(22) PCT Filed: Nov. 18, 2022
(86) PCT No.: PCT/KR2022/018252

§ 371 Date: May 31, 2024

(30) Foreign Application Priority Data
Dec. 13, 2021 (KR) 10-2021-0177767

(51) Int. Cl. A61K 35/30; A61P 27/02

(52) U.S. Cl. CPC A61K 35/30; A61P 27/02

(57) ABSTRACT

Provided is a method for preventing or treating optic neuropathy, comprising administering a pharmaceutical composition including neural progenitor cells as an active ingredient, where the neural progenitor cells not only exhibit remarkably superior neuroprotective and pro-regenerative effects compared to mesenchymal stem cells such as placenta-derived stem cells (e.g., PSCs), but also can be administered by non-invasive and safe subtenon injection, and therefore the method may be advantageously used as a cell therapy for treating optic neuropathy.

### TECHNICAL FIELD

The present invention relates to a pharmaceutical composition for preventing or treating optic neuropathy, comprising neural progenitor cells as an active ingredient.

### BACKGROUND ART

Because there is no effective therapy for irreversible damage to the optic nerve, many studies have attempted to improve the essential regenerative capacity of retinal ganglion cells (RGCs). Macrophage-activating factors and zymosan promote axon regrowth after optic nerve damage. Changes in the inherent renewal capacity of RGCs may be caused by deficiencies of phosphatase and tensin homolog (PTEN). The combination of inflammatory induction through injection of zymosan, PTEN deficiency, and promotion of intracellular cyclic adenosine monophosphate (cAMP) can help restore the optic nerve. However, approved treatments are difficult to use in clinical trials.

For incurable eye diseases, embryonic stem cells (ESCs), limbal stem cells, retinal pigment epithelial cells, and mesenchymal stem cells (MSCs) are used for cell therapy as part of regenerative medicine. MSCs attract immune cells by releasing immune modulators, mediators, and chemokines. In addition, MSCs have neuroprotective effects by secreting elements via paracrine action. In numerous studies that used animal models of glaucoma, the survival rate of RGCs increased when MSCs were injected intravitreally. In one study of an ischemic model, the number of RGCs and expressions of BDNF, ciliary neurotrophic factor (CNTF), and fibroblast growth factor (bFGF) after MSC injection were increased. Several studies have also identified the therapeutic effects of MSCs in ischemic models. In addition to MSCs derived from bone marrow, those derived from human umbilical cord blood, dental pulp, and placenta show therapeutic effects in terms of generative induction and axonal growth of damaged optic nerves (Kwon, H. et al., Hypoxia-Preconditioned Placenta-Derived Mesenchymal Stem Cells Rescue Optic Nerve Axons via Differential Roles of Vascular Endothelial Growth Factor in an Optic Nerve Compression Animal Model. Mol. Neurobiol. 2020, 57, 3362-3375; Labrador-Velandia, S. et al., Mesenchymal stem cell therapy in retinal and optic nerve diseases: An update of clinical trials. World J. Stem Cells 2016, 8, 376-383).

The present inventors previously reported that the regulation of hypoxia-inducible factor 1-alpha (Hif-1α) and growth-associated protein 43 (GAP43) of MSCs derived from human placenta (PSCs) promote axon survival in an optic nerve compression (ONC) model (Chung, S. et al., Human umbilical cord blood mononuclear cells and chorionic plate-derived mesenchymal stem cells promote axon survival in a rat model of optic nerve crush injury. Int. J. Mol. Med. 2016, 37, 1170-1180). In addition, regulation of the NF-Kb pathway plays an important role in the regulation of target proteins in PSCs. Another study demonstrated the possibility of using hypoxia-preconditioned PSCs (HPPCs) in cell therapy for optic nerve damage by examining the effects of regenerated nerves from HPPCs on R28 cells and in an animal model of optic nerve damage.

Many different molecules help regulate axon regeneration after optic nerve damage including growth factors such as Wnt, CNTF, BDNF, and semaphorins; growth-inhibitory transcription factors such as Kruppel-like factor 4 (KLF4); and essential signaling mediators such as Signal transducer and activator of transcription 3 (STAT3). The mechanisms by which Wnt signaling promotes axon regeneration may include induction of these axon growth-promoting genes; Wnt signaling may also directly control growth cone remodeling by changing microtubule stability during axonal growth.

### DISCLOSURE

### Technical Problem

The present inventors carried out various studies on the safety and clinical efficacy of human pluripotent stem cell-derived neural progenitor cells (NPCs). As a result, the present inventors have found that, in hypoxia-injured R28 cells and an optic nerve compression (ONC) model, NPCs show remarkably excellent neuroprotective and pro-regenerative effects compared to mesenchymal stem cells such as human placenta-derived stem cells (e.g., PSCs), thereby being able to be advantageously used as a cell therapy for treating optic neuropathy.

Accordingly, it is an object of the present invention to provide a pharmaceutical composition for preventing or treating optic neuropathy, comprising neural progenitor cells (NPCs) as an active ingredient.

### Technical Solution

In accordance with an aspect of the present invention, there is provided a pharmaceutical composition for preventing or treating optic neuropathy, comprising neural progenitor cells as an active ingredient.

The neural progenitor cells may be neural progenitor cells derived from human embryonic stem cells, preferably SOX1-positive and PAX6-positive neural progenitor cells derived from human embryonic stem cells.

The optic neuropathy may be one or more selected from the group consisting of ischemic optic neuropathy, optic neuritis, compressive optic neuropathy, infiltrative optic neuropathy, traumatic optic neuropathy, and mitochondrial optic neuropathy.

The pharmaceutical composition of the present invention may have a dosage form for subtenon injection.

### Advantageous Effects

It has been found by the present invention that, in hypoxia-injured R28 cells and an optic nerve compression (ONC) model, NPCs show remarkably excellent neuroprotective and pro-regenerative effects compared to mesenchymal stem cells such as human placenta-derived stem cells (e.g., PSCs). And, the pharmaceutical composition of the present invention can be preferably administered through a non-invasive, safe administration route, i.e., through subtenon injection. Therefore, the pharmaceutical composition of the present invention can be advantageously used as a cell therapy for treating optic neuropathy.

### DESCRIPTION OF DRAWINGS

FIG. 1 shows characterization of human pluripotent stem cell-derived neuronal progenitor cells (NPCs). FIG. 1A shows that CHA15 human ESCs were differentiated into NPCs by the treatments with 5 μM PKCβ inhibitor, and 1 μM DMH1 in the medium consisting of DMEM/F12, 10 μg/mL human insulin, 9 μg/mL transferrin, and 14 ng/ml selenite. FIG. 1B shows that the expanded NPCs at passage 1 were shown to be positive for two representative NPC markers, SOX1 (˜90%) and PAX6 (˜75.6%), but negative for a typical neural crest stem cell marker, P75 (˜0%). FIG. 1C shows that, when further differentiated into mature neurons, the NPCs generated both early and late neuronal markers, TUJ1 and MAP2, respectively.

FIG. 2 shows that human NPCs have the function of recovering damaged R28 cells. R28 cells were cultured with NPCs or hPSCs 3 hours prior to CoCl₂ treatment. Then, the R28 cells were treated with CoCl₂ (300 μM). FIG. 2A shows the viability assays performed after 24 hours. Data are expressed as percentage (mean±SEM) of viable cells compared to those in the control group. Significantly different values between groups are indicated by different letters (p<0.05). FIG. 2B shows the results obtained by determining apoptosis-related protein expressions. FIG. 2C shows Western blot analyses of target protein expression levels. The quantified values of target protein expression are presented (bottom panel) (*p<0.05 vs. the control; #p<0.05, ##p<0.01 vs. CoCl₂; †p<0.05 vs. PSCs).

FIG. 3 shows that the NPCs regulate axonal regeneration and inflammatory proteins in the retina and optic-nerve-injured rat model. Changes in target proteins were assessed by immunoblot analyses of rat retina and optic nerve extracts. The samples were analyzed 1, 2, and 4 weeks after injection with optic nerve compression. Expression levels were normalized to β-actin and the values of OS were divided OD. FIG. 3A shows quantified values of Hif-1α, Vegf, Neuroflament, NeuN, Thy-1 and Gfap expressions in retina extract. FIGS. 3B and 3C show quantified values of Bdnf, Iba1, Nlrp3 and Tnf-α expression in retina (B) and optic nerve tissues extract (C). The results are expressed as the mean±SEM of independent retina and optic nerve analyses, and are expressed as fold changes compared to the control (*p<0.05 vs. the age-matched sham (balanced salt solution, BSS): #p<0.05; ##p<0.01 vs. PSCs). OD, oculus dexter; OS, oculus sinister.

FIGS. 4a and 4b show the effects of NPCs promoting RGCs and axon regeneration in ONC models. A and B of FIG. 4a show the representative confocal microscopy-based fluorescence images following Brn-3a (A) and Tuj1 (B) staining (original magnification: 400×) of NPCs and hPSCs injection in the optic nerve compression animal model. C and D of FIG. 4b show the Gap43 (C) and Iba1 (D) fluorescence quantification performed at a 500 μm distance from the ONC site of the optic nerve. Total GAP43-positive cells were measured using ZEN software. Two retinas and optic nerves from each group were used. The results are presented as the mean±standard error of the mean (SEM) (*p<0.05 vs. the age-matched sham (balance salt solution): #p<0.05 vs. PSCs).

FIG. 5 shows Wnt/β-catenin and Nf-kb during recovery process of damaged R28 cells. After being co-cultured with NPCs or hPSCs, R28 were exposed to CoCl₂ (300 μM). After incubation for 24 hours, Western blotting analyses were performed. The results are expressed as the mean #standard error of the mean (SEM) (*p<0.05 vs. the control; #p<0.05 vs. CoCl₂).

### BEST MODE FOR CARRYING OUT THE INVENTION

The present inventors previously reported that human placenta-derived mesenchymal stem cells (PSCs) have neuroprotective effects. To evaluate the potential benefit of NPCs, we compared them to PSCs using R28 cells under hypoxic conditions and a rat model of optic nerve injury. NPCs and PSCs (2×10⁶ cells) were injected into the subtenon space. After 1, 2, and 4 weeks, we examined changes in target proteins in the retina and optic nerve. NPCs significantly induced vascular endothelial growth factor (Vegf) compared to age-matched shams and PSC groups at 2 weeks. NPCs also induced neurofilaments in the retina compared to the sham group at 4 weeks. In addition, the expression of brain-derived neurotrophic factor (Bdnf) was high in the retina in the NPC group at 2 weeks. The low expression of ionized calcium-binding adapter molecule 1 (Iba1) in the retina had recovered at 2 weeks after NPC injection and at 4 weeks after PSC injection. The expression of the inflammatory protein NLR family, pyrin domain containing 3 (Nlrp3) was significantly reduced at 1 week, and that of tumor necrosis factor-α (Tnf-α) in the optic nerves of the NPC group was lower at 2 weeks. Regarding retinal ganglion cells, the expressions of Brn3a and Tuj1 in the retina were enhanced in the NPC group compared to sham controls at 4 weeks. NPC injections increased Gap43 expression from 2 weeks and reduced Iba1 expression in the optic nerves during the recovery period. In addition, R28 cells exposed to hypoxic conditions showed increased cell survival when cocultured with NPCs compared to PSCs. Both Wnt/β-catenin signaling and increased Nf-κb could contribute to the rescue of damaged retinal ganglion cells via upregulation of neuroprotective factors, microglial engagement, and anti-inflammatory regulation by NPCs. Therefore, this study demonstrates that NPCs could be useful for the cellular treatment of various optic neuropathies.

Accordingly, the present invention provides a pharmaceutical composition for preventing or treating optic neuropathy, comprising neural progenitor cells as an active ingredient.

The neural progenitor cells (NPCs) are the progenitor cells of the CNS that give rise to many, if not all, of the glial and neuronal cells. NPCs are present in the CNS of developing embryos but are also found in the neonatal and mature adult brain. NPCs can be generated in vitro by differentiating embryonic stem cells or induced pluripotent stem cells (iPSC). The neural progenitor cells may be preferably neural progenitor cells derived from human embryonic stem cells, more preferably SOX1-positive and PAX6-positive neural progenitor cells derived from human embryonic stem cells.

The optic neuropathy refers to damage to optic nerve due to various causes. The characteristics of optic neuropathy are damage and death of nerve cells or neurons. Optic neuropathy is often referred to as an optic atrophy, which is the end point of disease that causes damages of nerve cells between retinal ganglion cells and lateral geniculate body. Therefore, in the pharmaceutical composition of the present invention, the optic neuropathy includes various forms of optic neuropathy, for example, may be one or more selected from the group consisting of ischemic optic neuropathy; optic neuritis; compressive optic neuropathy; infiltrative optic neuropathy; traumatic optic neuropathy; and mitochondrial optic neuropathy such as nutritional optic neuropathy, toxic optic neuropathy, hereditary optic neuropathy, and the like.

The pharmaceutical composition of the present invention may include a pharmaceutically acceptable carrier, for example, an emulsifier, a suspending agent, a buffering agent, an isotonic agent, and the like. The pharmaceutical composition of the present invention may be formulated to parenteral administration form. In particular, the pharmaceutical composition of the present invention may be preferably administered through a non-invasive and safe administration route, i.e. subtenon injection. Accordingly, the pharmaceutical composition of the present invention may have a dosage form for subtenon injection. The dosage form for subtenon injection may be prepared typically by preparing a sterile solution of the active ingredient; and may include a buffering agent that is capable of suitably adjusting the pH of the solution and an isotonic agent such that isotonicity is imparted to the formulation.

In addition, the pharmaceutical composition according to the present invention may be administered to a patient suffering from optic neuropathy once or repeatedly at a daily dose of about 1×10⁷ to 1×10⁸ cells, preferably about 5×10⁷ cells, which may be changed according to age and symptoms of the patient.

Hereinafter, the present invention will be described more specifically by the following examples and test examples. However, the following examples and test examples are provided only for illustrations and thus the present invention is not limited to or by them.

### 1. Materials and Methods

### (1) In Vitro Study

### (1-1) Human Pluripotent Stem Cell-Derived Neural Progenitor Cells (NPCs)

### Preparation

### Culture of Human Pluripotent Stem Cells

H9 human ESCs (WiCell Research Institute, Madison, WI, USA) were routinely maintained on Matrigel-coated culture dishes (BD Biosciences, San Jose, CA, USA) in TeSR™-E8™ medium (STEMCELL Technologies, Vancouver, BC, Canada). For passaging, ESCs were incubated with 0.5 mM EDTA (Thermo Fisher Scientific, Waltham, MA, USA) in a 37° C. CO₂ incubator for 3 minutes and then were split in the ratio of 1:20 onto Matrigel-coated dishes with TeSR™-E8™ medium containing 10 μM Y-27632 (Sigma-Aldrich, St. Louis, MO, USA). The medium was changed to TeSR™-E8™ medium without Y-27632 daily from two days after passage. Experiments using hESCs were approved by the Institutional Review Board of CHA University (IRB No. 1044308-201603-LR-004-09).

### Embryoid Body (EB) Formation and Induction of NPCs

Human ESCs were detached with 2 mg/ml collagenase Type IV (Worthington Biochemical Corporation, Lakewood, NJ, USA), for 30 minutes at 37° C. EBs were formed from the detached ESCs and were cultured in suspension for 4 days in DMEM/F12 (Thermo Fisher Scientific) containing 10 μg/mL human insulin, 9 μg/mL transferrin, 14 ng/ml selenite, 5 μM PKCβ inhibitor, and 1 μM DMH1 (all from Sigma-Aldrich). The culture medium was changed daily. On Day 4 of culture, EBs were transferred to Matrigel-coated dishes with NPC specification medium containing 1% N2 supplement (Thermo Fisher Scientific), 20 ng/ml bFGF (CHAbiotech, Pangyo, Korea), and 25 μg/mL human insulin (Sigma-Aldrich). The medium was changed every day for 5 days to generate neural rosettes.

### Flow Cytometry Analysis

Cells were dissociated into single cells with Accutase (Thermo Fisher Scientific) for 5 minutes at 37° C. and were fixed in 4% paraformaldehyde/phosphate-buffered saline (PBS) for 15 minutes at room temperature (RT). The fixed cells were treated with 0.2% Triton X-100 (Sigma-Aldrich)/PBS for 15 minutes at RT and incubated in blocking solution (1% BSA/PBS) for 30 minutes at RT. The cells were stained with anti-SOX1-PE, anti-PAX6-APC (all from Miltenyi Biotec GmbH, Bergisch Gladbach, Germany) overnight at 4° C. Since anti-p75-PE (Miltenyi Biotec GmbH) targeted a cell-surface antigen, p75, the permeabilization process was excluded. The isotype-matched IgG was used as a control. The cells were washed once in 1% BSA/PBS and analyzed using the CytoFLEX Flow Cytometer (Beckman Coulter, Brea, CA, USA).

### Immunocytochemistry

Cells were fixed in 4% paraformaldehyde/PBS and permeabilized in 0.2% Triton X-100 for 15 minutes each at RT. The cells were blocked with 5% BSA/PBS for 1 hour at RT and were treated with primary antibodies overnight at 4° C. The primary antibodies used were targeting TuJ1 (Covance, Burlington, NC, USA) and MAP2 (Millipore, Burlington, MA, USA), and the fluorescence-conjugated secondary antibodies used were Alexa Fluor 488 and Alexa Fluor 594, respectively (all from Thermo Fisher Scientific). The samples were treated with 4′,6-diamidino-2-phenylindole (DAPI) (Sigma-Aldrich) for 10 minutes after the secondary antibody treatment. Images were taken using a ZEISS fluorescence microscope (ZEISS, Oberkochen, Germany).

### (1-2) Human Placenta-Derived Mesenchymal Stem Cells (PSCs) Preparation

Human placenta stem cells were collected from Cha General Hospital in Seoul, South Korea. Sampling and use for research purposes were approved by the institutional review committee of the hospital. Preparation and culture operations were performed as previously reported (Park, M. et al., Human placenta mesenchymal stem cells promote axon survival following optic nerve compression through activation of NF-kappaB pathway. J. Tissue Eng. Regen. Med. 2018, 12, e1441-e1449).

### (1-3) Cell Culture and Treatment

The R28 retinal precursor cells were incubated in Dulbecco's minimal Eagle's medium (DMEM; Sigma-Aldrich) supplemented with 10% fetal bovine serum (FBS; Thermo Fisher Scientific), 1× minimal essential medium (MEM) with nonessential amino acids (Thermo Fisher Scientific), 100 μg/mL gentamicin (Sigma-Aldrich), and 1% penicillin-streptomycin (Thermo Fisher Scientific). Hypoxic condition was caused by the exposure of R28 cells to cobalt chloride (CoCl₂) (Sigma-Aldrich). R28 cells (2×10⁵) were seeded in a 6-well plate, and NPC or hPSC was co-cultured with R28 cells 3 hours prior to CoCl₂ treatment. Then, R28 cells were treated with CoCl₂ (300 μM), and the samples were prepared for experiment 24 hours later.

### (1-4) Cell Viability Assay

Cells were collected 24 hours after co-culturing of NPCs or PSCs (2×10⁵) with hypoxic R28 cells and calculated by microscopy. Cells were stained with trypan blue reagents and only cells identified as surviving cells were counted. The data are presented as the percentage of viable cells (means±SEMs) in the experimental group compared to the control group.

### (1-5) Immunoblot Analyses

Regenerative and inflammatory markers were analyzed using optic nerve tissue. Lysates were produced from optic nerve tissue using a PRO-PREP solution (iNtRON Biotechnology, Gyeonggido, Korea). The same amounts of total proteins were separated by SDS-electrophoresis and transferred to the membrane. The membranes were incubated with anti-Thy-1 (SC-53116), anti-β-actin (SC-47778) (Santa Cruz Biotechnology, Santa Cruz, CA, USA), anti-Vegf (GTX102643), anti-Tnf-α (GTX10520), anti-β-catenin (GTX101435), anti-Wnt3a (GTX128101), (GeneTex, Irvine, CA, USA), anti-GFAP (#3670), anti-Neurofilaments (#2837), anti-tCaspase3 (#9662), anti-Bcl2 (#2764), anti-Nf-κb (#8242) (Cell Signaling Technology, Danvers, MA, USA), anti-Hif-1a (PA1-16601), anti-Bdnf (PA5-85730), anti-Iba1 (PA5-27436) (Thermo Fisher Scientific), anti-Nlrp3 (NBP2-12446) (Novus Biologicals, Centennial, CO, USA), or anti-NeuN (MABB377) (Millipore) antibodies. All antibodies except Thy-1 (1:200 dilution) were used in a 1:1000 dilution ratio. After washing steps, the membranes were incubated with horseradish peroxidase-conjugated anti-rabbit or mouse secondary antibodies at a 1:10,000 dilution (GeneTex) for overnight at 4° C. Immuno-active bands were visualized as enhanced chemiluminescence solutions (Bio-Rad Laboratories, Hercules, CA, USA) and detected using ImageQuant™ LAS 4000 (GE Healthcare, Chicago, IL, USA).

### (2) In Vivo Study

### (2-1) Animals and the Study Group

Six-week-old male Sprague-Dawley (SD) rats (Orient Bio, Gyeonggido, Korea) were housed in standard animal facilities where food and water were provided at constant temperatures of 21° C. The in vivo experiment protocol was approved by the Institutional Animal Care and Use Committee of Bundang CHA Medical Center (IACUC200138). The rats were classified into the following groups: Sham (balanced salt solution (BSS) injection after optic nerve compression); NPC group (2×10⁶/0.06 mL injection after optic nerve compression); PSC group (2×10⁶/0.06 mL injection after optic nerve compression). After 1, 2, and 4 weeks, the animals were euthanized.

### (2-2) The Optic Nerve Compression Model and Subtenon Cell Injection

The rats were anesthetized by Zoletil and Rompun. Animal model production was carried out as mentioned in the previous study (Chung, S. et al., Human umbilical cord blood mononuclear cells and chorionic plate-derived mesenchymal stem cells promote axon survival in a rat model of optic nerve crush injury. Int. J. Mol. Med. 2016, 37, 1170-1180). After locally applying 0.5% proparacaine hydrochloride, lateral canthotomy and conjunctival incision were performed. Tissues enclosing the optic nerve were dissected. Using ultra-fine self-closing forceps, optic nerve was compressed at 2 mm site behind the globe for 5 seconds. Optic nerve compression (ONC) was performed in the left eye (oculus sinister; OS). Then, the canthal incision was sutured. After thorough suturing of the canthal site, subtenon injection of NPCs or PSCs into the nasal side of the eyeballs of the rats was performed.

### (2-3) Assessment of Axon Regeneration Factors in the Optic Nerve of ONC Model

For in vivo measurement of axon regeneration, the vertical portion stained with GAP43 of the optic nerve was photographed. The optic nerves were fixed with 4% paraformaldehyde and embedded in paraffin. The optic nerve was cut vertically to a thickness of 20 μm and mounted on glass slides. Anti-GAP43 antibody (1:200, ab75810; Abcam, Cambridge, UK) or anti-Iba1 antibody (1:200, PA5-27436, Thermo Fisher Scientific) was used to stain the regenerating fibers. The measurement site was a rectangular area of W 150 μm×H 700 μm on both sides of the ONC area, followed by computation of the mean. Total GAP43 or Iba1-positive cells were determined using ZEN software (Carl Zeiss, Jena, Germany).

### (2-4) Flat-Mounted Retinas and RGC Survival Analyses

After the enucleation of three rats from each treatment group, the retina was dissected with a flattened whole mount. After taking out of the cornea by cutting a circular path along the ora serrata with small scissors, the lens was removed using forceps. Separation of the retina from the eyecup was performed by placing forceps between the retina and the eyecup. After obtaining the entire retina, it was cut into quarters using scissors to incise from the retinal to the optic nerve, as described in previous study (Kwon, H. et al., Hypoxia-Preconditioned Placenta-Derived Mesenchymal Stem Cells Rescue Optic Nerve Axons via Differential Roles of Vascular Endothelial Growth Factor in an Optic Nerve Compression Animal Model. Mol. Neurobiol. 2020, 57, 3362-3375). The retinas were fixed in 4% paraformaldehyde and mounted on a glass coverslip for at least 1 hour at room temperature. After washing with PBS, they were incubated in PBS with 1% Triton X-100 at room temperature for 30 minutes. The retina was blocked in 20% fetal calf serum for 1 hour, and incubated with anti-Tuj1 antibody (ab18207; Abcam) or anti-Brn-3a (MAB1585; Millipore) antibody at a 1:10 dilution for overnight at 4° C. On the next day, the retina was washed with PBS-T and incubated with goat anti-rabbit IgG-fluorescein isothiocyanate and Alexa Fluor 633 antibodies in PBS-T at 1:200 for 2 hours. Before mounting on the coverslip, retina was washed again. Images captured using a confocal microscope (LSM 880; Carl Zeiss, Jena, Germany) were used to quantify fluorescence. Two areas were calculated on each retina, and the average values were compared for statistical analysis.

### (3) Statistical Analyses

Data analyses were conducted using GraphPad Prism9 (GraphPad, La Jolla, CA, USA). Statistically significant differences were identified using the t-test or nonparametric statistical test, followed by the Mann-Whitney U test at a significance level of 5%.

### 2. Results

### (1) Characterization of Human Pluripotent Stem Cell-Derived Neuronal Progenitor Cells (NPCs)

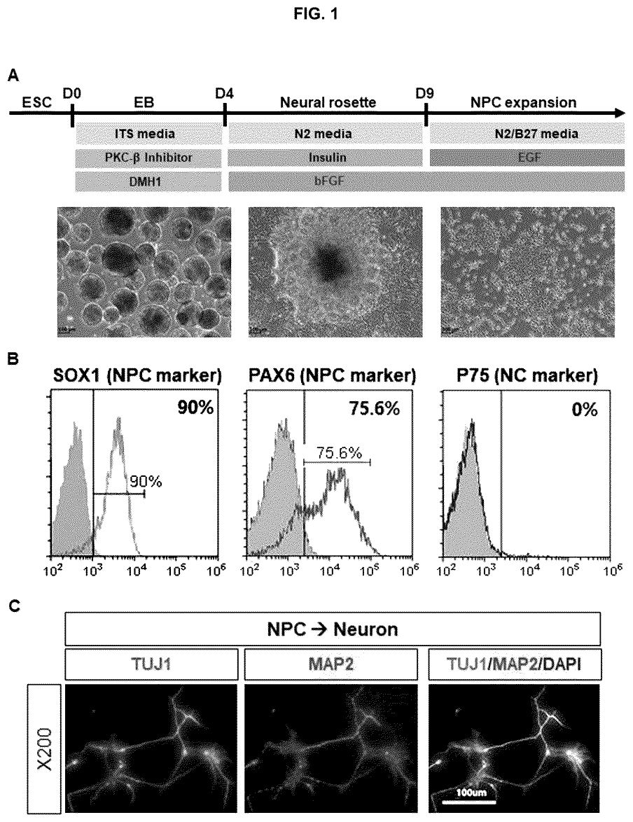

CHA15 human ESCs were differentiated into NPCs by treatments with 5 μM PKCβ inhibitor, and 1 μM DMH1 in the medium consisting of DMEM/F12, 10 μg/mL human insulin, 9 μg/mL transferrin, and 14 ng/ml selenite (FIG. 1A). Expanded NPCs at passage 1 were positive for two representative NPC markers, SOX1 (˜90%) and PAX6 (˜75.6%), but negative for a typical neural crest stem cell marker, P75 (˜0%) (FIG. 1B). When further differentiated into mature neurons, the NPCs generated both early and late neuronal markers, TUJ1 and MAP2, respectively (FIG. 1C).

### (2) NPCs Reduce Cell Apoptosis and Regulate Target Proteins

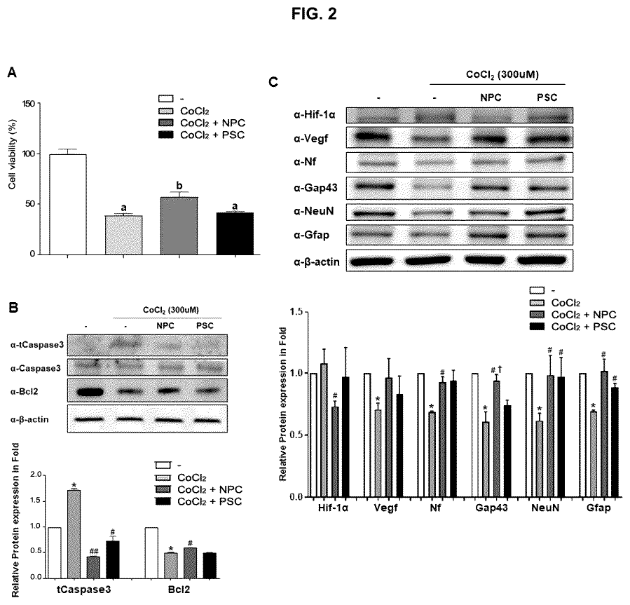

To assess the recovery function of NPCs, we performed cell viability tests. When cocultured with NPCs or PSCs, the viability of damaged R28 cells recovered by 48% and 7% more, respectively, than under hypoxic conditions (FIG. 2A). In addition, NPCs regulated cleaved caspase-3 activity and Bcl-2 protein expression during apoptosis (FIG. 2B). We also found that Hif-1α cocultured with PSCs showed less hypoxic damage. In contrast to Hif-1α, decreased expressions of neurofilaments (Nf), Gap43, NeuN, and Gfap under hypoxic conditions were significantly recovered by coculturing them with NPCs (FIG. 2C).

(3) Changes in Neurogenic Marker Expression in the Retina after Injection of NPCs or PSCs in the Optic Nerve Compression Animal Model

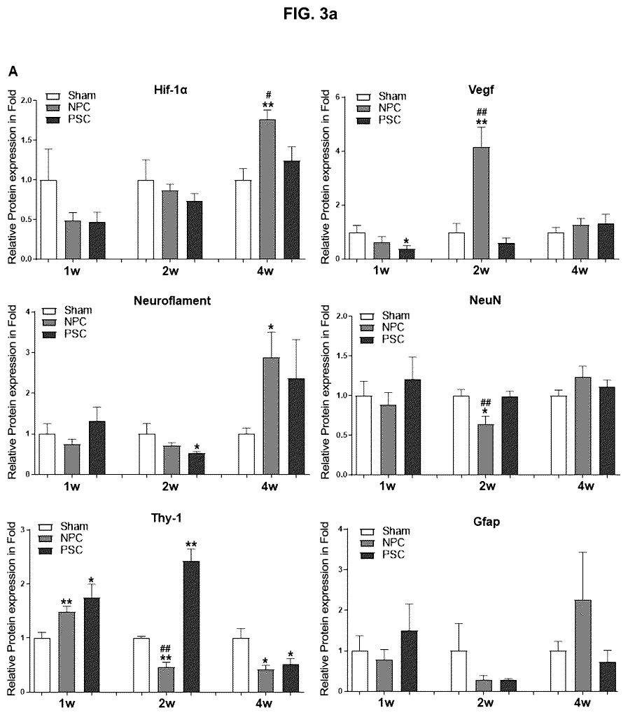

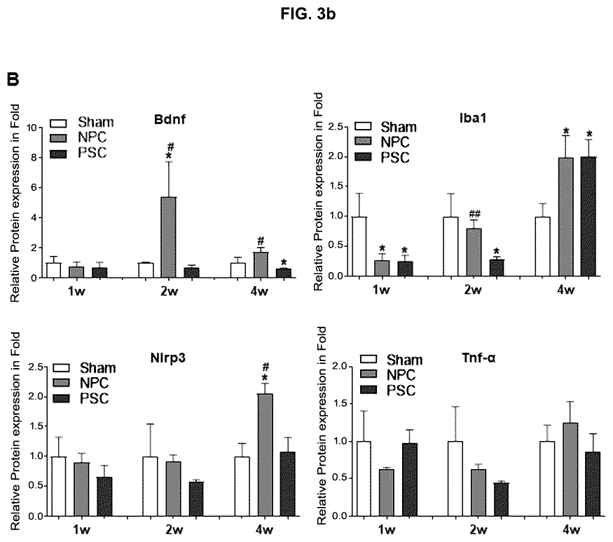

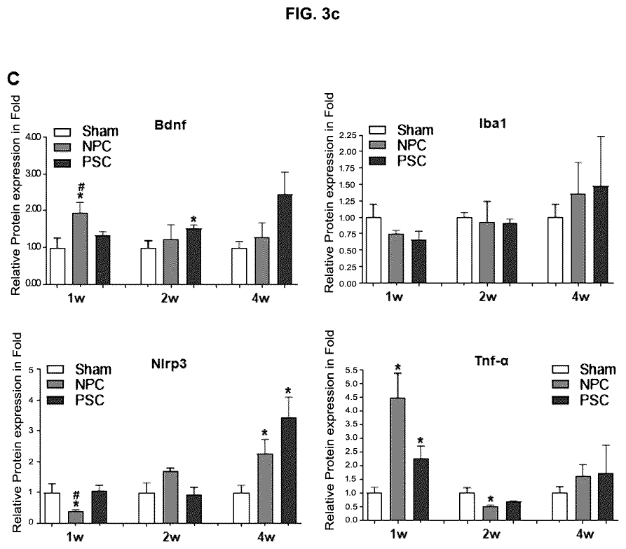

Regulation of the expressions of Hif-1α, Vegf, Neurofilaments, NeuN, Thy-1, and Gfap proteins in the rat retina was analyzed by Western blotting at 1, 2, and 4 weeks after optic nerve compression. At 1 week, Thy-1 expression was significantly increased by NPCs and PSCs compared to the age-matched sham group. NPCs significantly induced Vegf in the retina compared to the sham group and PSC group. NPCs also increased Neurofilament induction compared to the sham group at 4 weeks (FIG. 3A).

### (4) Comparison of the Target Protein Expression Between the Retina and the Optic Nerve Tissue in the Optic Nerve Compression Model

We compared the expressions of target protein in the retina versus optic nerve using the ONC model. BDNF expression in the retina was high in the NPC group at 2 weeks, while expression in the optic nerve was high in both the NPC and PSC groups. For Iba1, the lowered expression in the retina was recovered 2 weeks after NPC injection and at 4 weeks after PSC injection (FIG. 3B). The expression of the inflammatory protein Nlrp3 was significantly reduced at 1 week and that of Tnf-α was significantly reduced at 2 weeks in the optic nerves of the NPC group (FIG. 3C).

### (5) Protective Effects of NPCs and PSCs on the Retinal RGCs in the Optic Nerve Compression Animal Model

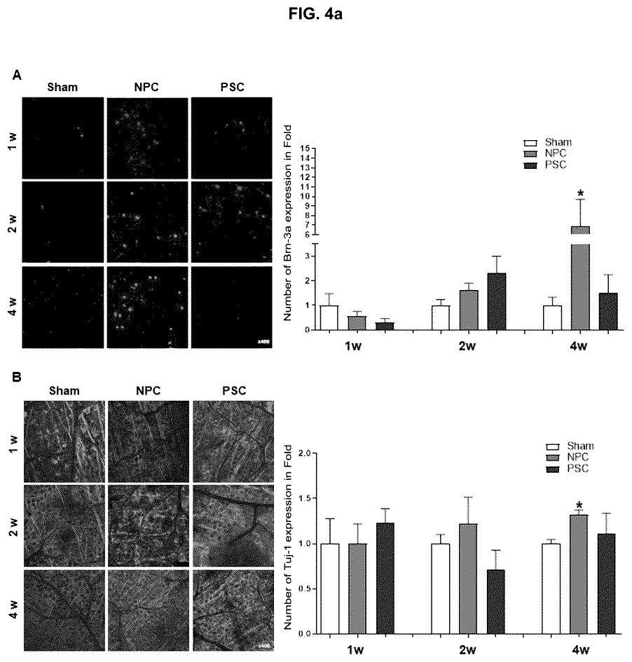

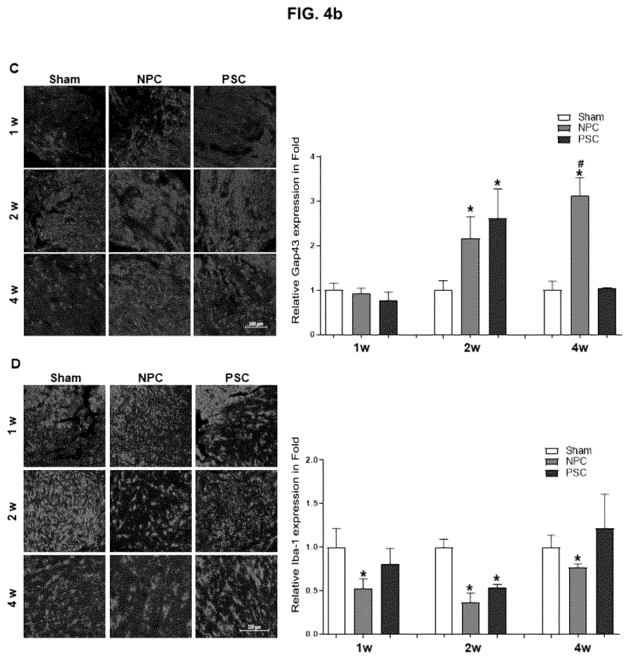

We assessed the survival rates of RGCs by counting the numbers of RGCs stained with Brn-3a and Tuj1 in rat retinas. After ONC, we found that only NPCs significantly increased Brn-3a and Tuj1 expression more than that in the age-matched sham group in the retina at 4 weeks (A and B of FIG. 4a).

### (6) Effects of NPCs and PSCs on Optic Nerve Axon Damage in the Optic Nerve Injury Animal Model

We assessed the protective effects of NPC injection on optic nerves by calculating the GAP43 and Iba1-positive cells from the optic nerve of ONC models. As shown in C of FIG. 4b, the expression of GAP43 was significantly increased at 2 weeks in both treatment groups, compared to the ONC group, while only the NPC injection showed significant recovery at 4 weeks. In addition, NPC injection reduced the expression of Iba1 in the optic nerves for 4 weeks, indicating that they could promote microglial enrollment to the retina during the recovery period (D of FIG. 4b).

### (7) Wnt/β-Catenin Signal is Involved During Recovery of Retinal Ganglion Cells by NPCs

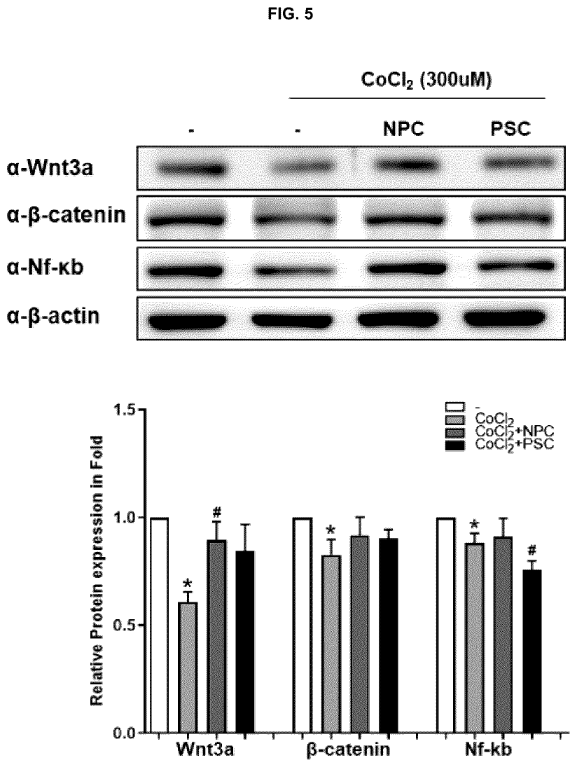

In previous report, it was reported that Wnt/β-catenin signaling and NF-Kb protein are involved in the neuroprotection by MSCs (Dvoriantchikova, G. et al., Virally delivered, constitutively active NFkappaB improves survival of injured retinal ganglion cells. Eur. J. Neurosci. 2016, 44, 2935-2943; Liu, X. et al., Interaction of NF-kappaB and Wnt/beta-catenin Signaling Pathways in Alzheimer's Disease and Potential Active Drug Treatments. Neurochem. Res. 2021, 46, 711-731). The present inventors examined whether Wnt/β-catenin signaling may be involved with the recovery mechanisms by NPCs of damaged R28 cells. We found that Wnt/β-catenin signaling mediated the NPC induced recovery process. When R28 cells were cocultured with NPCs, the CoCl₂-induced reduction of Wnt3a was significantly recovered compared to controls. In addition, the levels of NF-κb expression were maintained similar to normal in the NPC group, while the levels of NF-κb expression were lower than normal (and the NPC group) in the PSC group (FIG. 5).

### 3. Discussion

MSCs are easy to harvest from body fat, bone marrow, placenta, and umbilical cord. In addition, they are immune-privileged because they express low levels of HLA class I antigens and do not present, or present very low levels of, CD80, CD86, CD40, and HLA class II antigens. Furthermore, other unique features such as easy separation, rapid growth after a short period of dormancy, and exemption from ethical issues make MSCs useful for cell therapy. However, unlike MSCs such as PSCs, NPCs were derived from various conditions and chemical induction in many experiments (Kim, H. M. et al., Fine-tuning of dual-SMAD inhibition to differentiate human pluripotent stem cells into neural crest stem cells. Cell Prolif. 2021, 54, e13103).

The mechanisms by which MSCs directly regulate neuroprotection remain unclear. PSCs have neuroprotective effects via the moderation of Hif-1α and GAP43 (Chung, S. et al., Human umbilical cord blood mononuclear cells and chorionic plate-derived mesenchymal stem cells promote axon survival in a rat model of optic nerve crush injury. Int. J. Mol. Med. 2016, 37, 1170-1180) and mediation of NF-κb pathways (Dvoriantchikova, G. et al., Virally delivered, constitutively active NFkappaB improves survival of injured retinal ganglion cells. Eur. J. Neurosci. 2016, 44, 2935-2943); Liu, X. et al., Interaction of NF-kappaB and Wnt/beta-catenin Signaling Pathways in Alzheimer's Disease and Potential Active Drug Treatments. Neurochem. Res. 2021, 46, 711-731). These results suggest that MSC-based therapy may be useful for treating optic nerve disorders, but the critical pathway for optic nerve recovery is unclear.

In this study, the present inventors investigated the mediator proteins and pathways of NPCs associated with the recovery process of the RGC precursor cells. The expressions of Gap43, Thy-1 and neurofilaments, markers of neuronal regeneration, were induced by NPCs. The present inventors confirmed the functions of NPCs in vivo and in vitro. The neuroprotective and pro-regenerative effects of NPCs were remarkably superior to those of PSCs. To compare the therapeutic roles of PSCs and NPCs, we conducted BDNF-mediated functional analyses. BDNF expression in the retina was significantly high in the NPC group, while the expression in the optic nerve was high in both the NPC and PSC groups. These results show that NPCs are effective in the recovery of optic ganglion cells, where optic nerve axons originate.

In the present study, PSCs and NPCs were associated with Iba1 protein expression. NPCs induced microglial expression of Iba1 in the retina more than did PSCs during the recovery period. After optic nerve injury, activated microglia was observed to migrate from the optic nerve to the retina and participate in the cellular response in the damaged retina for the survival and clearance of the axons (Heuss, N. D. et al., Optic nerve as a source of activated retinal microglia post-injury. Acta Neuropathol. Commun. 2018, 6, 66). The expression of Iba-1, a microglia biomarker, is associated with microglia polarization. During the neuroprotection process, a microglial switch from M1 to the M2 phenotype was induced (Cui, W. et al., Inhibition of TLR4 Induces M2 Microglial Polarization and Provides Neuroprotection via the NLRP3 Inflammasome in Alzheimer's Disease. Front. Neurosci. 2020, 14, 444). Electroacupuncture enhanced the activations of M2 marker Arginase 1 (Arg1) and Iba1-positive cells in the hippocampus, when used as a treatment for neurodegenerative diseases such as Alzheimer's disease (Xie, L. et al., Electroacupuncture Improves M2 Microglia Polarization and Glia Anti-inflammation of Hippocampus in Alzheimer's Disease. Front. Neurosci. 2021, 15, 689-629). The induction of Iba-1 expression in retina may contribute to the expression of M2 biomarkers during neuroprotection after optic nerve injury. Thus, NPCs may be more efficient for neuronal rehabilitation after hypoxic injury. Especially, considering that the recovery effect of NPCs was more associated with microglial neuroprotection, NPCs can rescue damaged RGCs through cellular interactions better than PSCs.

The type of eye disorder would determine the routes of stem cell administration. For retinal diseases such as retinitis pigmentosa. Stargardt's disease, and age-related macular degeneration, intravitreal injection is conventionally used. The present inventors injected PSCs or NPCs via the subtenon route, which is less invasive and safer for repeated injections, compared to other routes such as intravenous or intravitreal. The subtenon injection of NPCs maintained a longer-lasting effect, compared to the injection of the same numbers of PSCs.

In conclusion, NPCs had beneficial effects on hypoxia-injured R28 cells and in an animal model of ONC. NPCs may rescue the damaged RGCs via upregulation of neuroprotection factors, microglial engagement, and anti-inflammatory regulation mediated by Wnt/β-catenin signaling and Nf-κb. Therefore, NPCs may be used as a useful cellular treatment for various optic neuropathies.

1. A method for preventing or treating optic neuropathy, comprising administering a pharmaceutical composition comprising neural progenitor cells as an active ingredient to a subject in need thereof.

2. The method according to claim 1, wherein the neural progenitor cells are neural progenitor cells derived from human embryonic stem cells.

3. The method according to claim 1, wherein the neural progenitor cells are SOX1-positive and PAX6-positive neural progenitor cells derived from human embryonic stem cells.

4. The method according to claim 1, wherein the optic neuropathy is one or more selected from the group consisting of ischemic optic neuropathy, optic neuritis, compressive optic neuropathy, infiltrative optic neuropathy, traumatic optic neuropathy, and mitochondrial optic neuropathy.

5. The method according to claim 1, wherein the pharmaceutical composition is a dosage form for subtenon injection.
